(19) United States
(12) Patent Application Publication
(10) Pub. No.: US 2025/0122584 A1
(43) Pub. Date: Apr. 17, 2025

(54) METHOD FOR DETECTION OF MICROORGANISMS OR GENE DEFECTS

(71) Applicant: Mikkel Ante Glomsrød, Bergen (NO)

(72) Inventor: Mikkel Ante Glomsrød, Bergen (NO)

(21) Appl. No.: 18/378,912

(22) Filed: Oct. 11, 2023

(51) Int. Cl. C12Q 1/689; C12Q 1/44; C12Q 1/48; C12Q 1/6883

(52) U.S. Cl. CPC C12Q 1/689; C12Q 1/44; C12Q 1/485; C12Q 1/6883

(57) ABSTRACT

Method for detection of a microorganism in an organic sample in a container where a CRISPR/Cas system cuts a predefined part of the microorganism's genome, if the microorganism we seek to detect is present in the sample, characterized by a nucleotide sequence coding for a reporter molecule with a partially complete promoter element/start codon, ligated with the cut part of the microorganism's genome resulting in a complete promoter element or start codon producing mRNA by RNA polymerase, resulting in an amino acid chain which may be detected and verifying the presence of the microorganism in the sample.

### BACKGROUND OF THE INVENTION AND PURPOSE

### 2.1 Introduction

The invention is a new method to detect a microorganism (bacteria, virus, fungus, etc.) in an organic sample, in a way that diagnosis of infectious diseases make take place outside of a laboratory in a quick and cost efficient way by use of pre-adjusted test equipment. The method can also be used for detection of genetic disorders such as Huntington's disease by performing biopsy and testing for correct genetic sequence. A negative test result would in that case implies detection of gene defect.

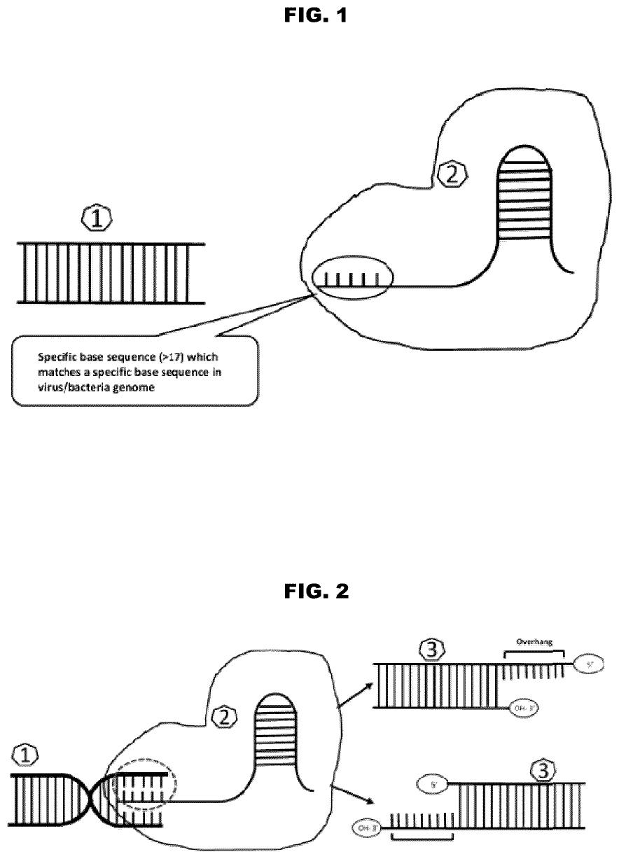

CRISPR (Clustered Regularly Interspaced Short Palindromic Repeats) is a method for gene editing which makes it possible to cut a predefined part of genetic material (DNA) (FIGS. 1 and 2). The most common application of CRISPR uses the Cas9-enzyme, and an RNA sequence (guide RNA) that recognizes or guides to the location where CRISPR shall cut in the DNA.

Is it prior art to use CRISPR to diagnose infectious diseases. However, a challenge with prior art is that it is necessary with laboratory capacity/special equipment to analyse samples and diagnoses. The special with the invention is that CRISPR combines with production capacity of CPFS (Cell Free Protein Synthesis).

The invention entails making of diagnosis equipment/test containers for one or more predefined microorganisms. By adding an organic sample (a practice a bodily fluid like spit or blood) to the diagnosis equipment/test container, the Invention can verify if the microorganism is present in the sample quicker and more efficiently than existing techniques.

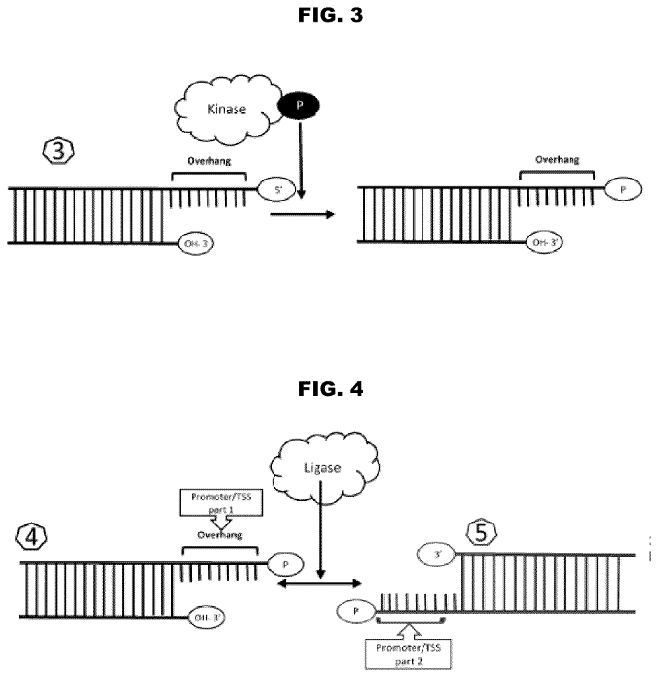

Verification happens when the cut part of the microorganism DNA gets phosphorylated (FIG. 3) and thereafter is ligated to DNA for a reporter molecule (FIG. 4). The method of ligase used is NHEJ (non-homologous end joining).

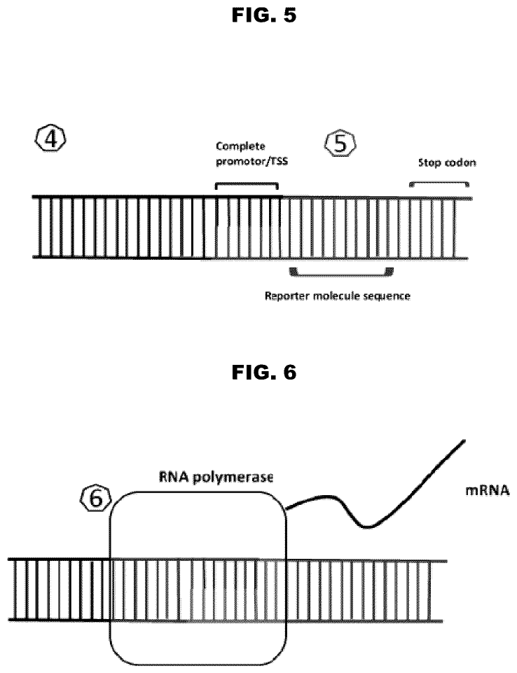

A specific base sequence at the end of each DNA strand will, after ligation of the two DNA segments, form a complete start codon. The ligated DNA sequence of the viral/bacterial DNA and the reporter molecule DNA combine to form a complete promoter/TSS, which is followed by a reporter molecule sequence and ends in a stop codon (FIG. 5). This again results in the amplification through RNA polymerase, which produces mRNA for further protein synthesis of a reporter molecule (FIG. 6).

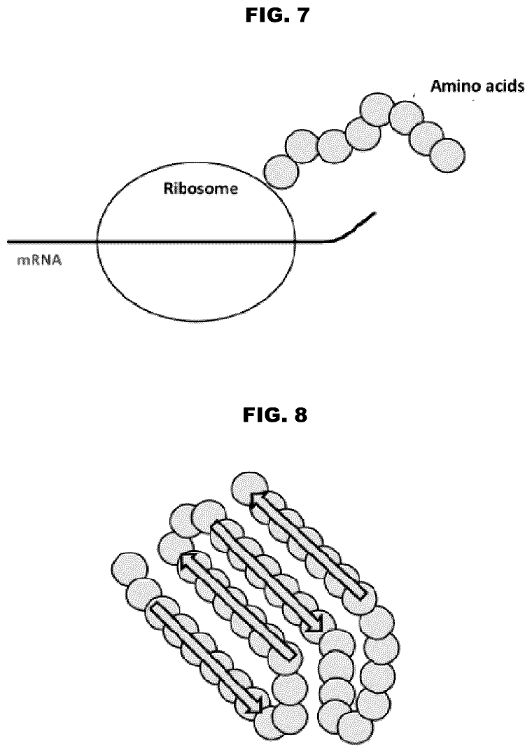

Ribosomes link to mRNA sequences which code for reporter molecules (FIG. 7). After linking, the ribosome produces amino acid chains that, either alone or with help of chaperones, will fold itself into a functional protein (FIG. 8).

The main reason that the invention works as a detection method is that the amplification process will not start unless the genetic sequence that is tested is present in the sample. A false positive result is very unlikely since the guide RNA (+/−20 base pairs) must match precisely with the sequence for disease before the procedure continues. The likelihood of this happening by accident is 5,8*10⁻¹¹% (mathematically calculated based on a 17 bp long guide RNA). Since the promoter/TSS for the sequence coding for the reporter molecule is not complete before it ligates with the genetical sequence it is tested for (which gives the matching overhang, cf, FIG. 4), the protein synthesis cannot start unless the microorganism that is being tested is present in the sample. This means that if reporter molecules have been produced, it is for sure a positive test result.

A positive test result is detected by a colour change or fluorescense in the test, as a result of the protein synthesis. Whether the result is verified by a colour change or fluorescense depends on the choice of reporter molecule.

This Invention and its processes is hereafter referred to as CAPS (Cas edited protein synthesis).

### 2.2 Further Description of the Invention

The procedure can be divided into three distinguished phases: sequence match, amplification, and observation.

In the first phase, the sample to be tested for a specific microorganism (or gene defect) is added to a capsule containing the test itself. The sample can be saliva, blood or other bodily fluids where the microorganisms which are tested may be present.

When the sample is added to the test capsule/container, a detergent will firstly break down the cell membrane (alternatively the capsid) and the DNA will be released. The DNA sequence, unique for the microorganism we seek to detect, can be picked up by a CRISPR-Cas complex, where the sequence which is being matched is singled out by aid of a RNA-molecule called sgRNA (single-guide RNA). When a unique match has been made, the Cas-complex will cut the specific disease sequence and leave an “overhang” that then will only match a reporter molecule DNA sequence already present in the test. This will hence complete a promoter sequence since the “overhangs” match that these two sequences may ligate and synthesis may start. The length of the sequence to be matched can vary between different Cas-complexes, but it is often between 17-25 bases long. The probability for 17 bases to be found randomly in a sample is close to zero (5,8*10⁻¹¹%). CRISPR/Cas is therefore acknowledged globally as a very precise tool.

There are two reasons why the synthesis of the reporter molecule cannot take place without presence of the disease sequence:
RNA polymerase is required to couple to a certain amount of base pairs upstream a promoter (before the sequence coding for the reporter molecule) to be able to produce mRNA
The promoter itself will not be complete before ligation to the disease sequence.

The next main phase is amplification, which happens in four steps.

The first step is via transcription, where DNA is translated to mRNA.

Amplification in this step is performed using RNA polymerase (RNAP) which reads the spliced viral/bacterial reporter molecule DNA sequence, and creates a mRNA for further protein synthesis. This amplification can be controlled/guided through gradient/amount of added RNA polymerase. With a higher gradient, the probability for the same DNA thread to go through the same procedure again with a new RNAP increases.

The next amplification step can be necessary for overall functionality. By using a DNA sequence which contains several instances of the same reporter molecule sequence, one after the other, another version of Cas (Cas13) can be used to cut the one long sequence into e.g. 10 mRNA sequences, each coding for one reporter molecule. One thus have 10 mRNA sequences from one round of transcription. The goal is to increase the concentration of mRNA for a reporter molecule to create a stronger signal which can be detected without detection equipment.

The next amplification step is the protein synthesis, where every mRNA can be used/transcribed many times by ribosomes. By having a high gradient of ribosomes present, it would be possible to control the reaction rate.

The last amplification step is connected to the choice of reporter molecule. One may either use a reporter molecule which itself can be detected in that it gives a clear colour change or one use a reporter molecule which then causes a cascade reaction. This may create one extra amplification step, which can be adjusted as necessary. The principle of this last amplification step will anyway be the same in that one may detect the present of a reporter molecule through colour change in the sample.

One example of a possible reporter molecule is β-galactosidase combined with X-gal. β-galactosidase, which is only produced through CFPS if guideRNA matches the disease sequence, will continuously break the bonds in X-gal by enzyme catalyzed hydrolysis to create a strong, blue colour. This amplifies the signal further. Other possible reporter molecules can be GUS (beta-glucoronidase) eller XyIE (catechol dioxygenase).

With a four-step amplification after the two DNA strands have been ligated one can see results without using instruments, and one can therefore use the test outside a laboratory, Since all steps happens through Cas edited protein synthesis (CAPS), the only thing needed from the person which is going to use the test is for instance to spit and wait a certain amount of time. The test can therefore be performed without professionals.

There are several patents based on CRISPR for detection of microorganisms, inter alia “Sherlock” (WO2018170340A1). However, the novelty of the invention is the combination of CRISPR with the matching of chosen sequence from reporter molecule which ligates by ligases and ATP, plus the above mentioned amplification processes. The difference of the Invention and prior art is Illustrated In the following table:

| | Known | |
|---|---|---|
| Invention | methods/techniques | Comments |
| A detergent breaks down cell | Common step for most | Chemicals breaking |
| walls and release DNA/RNA | tests using DNA/RNA, | up lipid |
| | but is performed in a | membranes, |
| | separate step in | possibly enzymes. |
| | laboratories. | |
| A Cas complex (guide RNA) is | SHERLOCK and | DNA/RNA exposed/ |
| connected to a unique disease | DETECTR connects to a | Guide RNA is |
| sequence (DNA/RNA) and cuts | disease sequence and | unique for each |
| at a specific point (to the right | modify already present | disease/defined |
| of the chosen sequence) which | reporter molecules | unique disease |
| is dependant upon a match | | sequence length will |
| with gRNA and that a PAM | | vary a lot from one |
| sequence (protein adjacent | | disease to another. |
| motif) is present. | | |
| A Cas complex cuts the | New compared to | Since we are free to |
| disease sequence at a specific | known techniques. | choose the location |
| point, resulting in base pairs | | in a high number of |
| which can complete a start | | base pairs, finding a |
| codon in combination with the | | start-point which |
| DNA sequence of our choice. | | suits our needs is |
| | | not a problem. |
| Matching of the known | New compared to | The end sequence |
| sequence of the reporter | known techniques. All | of the reporter |
| molecule ligates with use of | other methods use a | molecule matches |
| ligase and ATP. The ligase | modification of already | with the end |
| method used is NHEJ (non- | present reporter | sequence of the |
| homologues end joining). | proteins or cut | disease DNA. The |
| | DNA/RNA reporter | overhangs of the |
| | molecule hybrids and | reporter molecule |
| | measure the length | sequence results |
| | thereafter (both | that the translation |
| | SHERLOCK and | does not happen |
| | DETECTR). | unless the disease |
| | | sequence is |
| | | present. |
| Polymerase begins | Unique in combination | Reporter molecule |
| translation > produces | with the remaining | can be chosen as |
| mRNA which further | steps, from step six | needed (β- |
| produces a desired | and out. | galactosidase is |
| reporter molecule. | | reommended). |
| The increased signal is caused | Unique in combination | A possibility for |
| by CFPS which continuously | with the remaining of | Increasing signal is |
| produces molecules which can | elements/steps. | that reporter |
| be observed without technical | | molecule contains |
| aid (can be seen by the | | several |
| “naked eye”). There are three | | interconnected code |
| possibilities for amplification: | | sequencies, which |
| Produce more mRNA | | again will be cut by |
| sequences per DNA sequence. | | CRISPR −> which |
| Control/regulate | | further produces |
| concentration of RNA | | desired protein. |
| polymerase and ribosomes as | | Other possibilities |
| needed. | | involve enzymatic |
| The molecules produced can | | cascade reactions. |
| be an enzyme or cofactor of | | |
| an enzyme which will start a | | |
| cascade reaction. | | |
| Detection can either happen | Unique in combination | If everything goes |
| through the “naked eye” or | with the remaining of | as it should, this |
| through a detector depending | elements/steps. | may be detected |
| on whether the customer's | | with the naked eye. |
| need is a one-off or for | | But the price per |
| repeated use. | | sample may be |
| “The price of reagents vs. the | | adjusted in view of |
| price of the detector with | | a cheaper sensor, |
| lower concentration of | | optionally for |
| reagents.” | | household. |

### 2.3 General Information about Detergents, CRISPR and Protein Synthesis

### 2.3.1 Detergents

A detergent, in biological terms, is a substance which can break up cell membranes. This is done in that the detergent connects to phospholipid-membranes, cell walls, to subsequently replace and displace the membrane's phospholipids. This happens because the detergent has higher affinity to the cell wall than the original phospholipids. The cell membrane's phospholipids will then form micelles, which is an aggregation of lipids where phospholipids packs together. As a consequence of this, the cell wall will burst or create holes as a result of shortage of phospholipids. The cell wall will break, and the cell content, including the DNA we are seeking to test using the invention, will leak and can then be picked up by the CRISPR-Cas-Complex.

Choice of detergent in the execution of the invention will depend on the type of cell wall, that is which microorganism, that is of interest. It would further be advantageous to choose a detergent which is not harmful for other components needed to synthesise reporter molecules.

It should be mentioned that this is known technology and a wide range of possibilities exists.

### 2.3.2 The CRISPR-Cas Complex

The CRISPR-Cas system is originally a part of the immune system in bacteria and some archaea. It is later discovered that one could program the system to function as an RNA-guided endonuclease. Endonuclease is a group of enzymes which can cleave phosphodiester bonds in polynucleotide chains. This means that one can cut a DNA exactly where desired and edit DNA by ligating with another desired DNA sequence to e.g. produce a new product (in our case a DNA sequence for reporter molecule).

There are five main groups in the CRISPR family. Below, typeII/Cas9 is described as an example. CRISPR RNA (crRNA) interacts with «trans-activating crRNA» (tracrRNA) and forms a duplex. This complex tells where the cas9 should cut the DNA helix. This duplex can be replaced with a “single guide RNA”, which makes it possible to determine where the Cas complex will cut. The sequence “chosing” the cut location is usually around 20 base pairs long and must be in proximity of a “protospacer adjacent motif” (PAM). Cas9's PAM is NGG (X/Guanine/Guanine) or NAG (X/Adenosine/Guanine)-(which is a bit less effective), where N is any nucleotide. Even If PAM must be chosen selectively, there are many possibilities, since PAM is often not very specific and one may choose a PAM based on the gRNA sequence. The sensitivity of CRISPR comes from the length of gRNA, since the sequence must be an exact match for the Cas complexity to cut and the chance that a matching sequence is present by is 5,8*10⁻¹¹% (a probability calculation based on a 17 bases).

The Cas complex used in this method is called Cpf1, chosen for its property to create an “overhang” when it cuts, but is otherwise relatively similar to Cas9.

### 2.3.3 CFPS

Cell free protein synthesis (CFPS) is a method where a well-working “protein machinery” is taken out of a cell, cleaned and afterwards used to produce desired proteins. This can be done by giving the “machinery” DNA which codes for the desired proteins. The machinery is complete with transcription and translation.

CFPS is a relatively old technology which have created a lot of opportunities to express proteins. The technology produces proteins by adding a DNA template, energy (in the form of ATP), salts, cofactors and substrates to an already isolated cell extract, which has been prepared in a growth medium and thereafter exposed to cell lysate techniques and processing.

The advantages with CFPS is that one is not dependent upon living cells and can therefore focus all of the system's energy on producing the desired protein. The system may have higher efficiency than transfected bacteria, since bacteria will distribute energy to other tasks than protein synthesis.

The possibility to adjust production components and the environment will be essential for the optimal production of the reporter molecule. Many different versions of the CFPS have been developed over the last sixty years, the most commonly used are: Escherichia coli, Spodoptera, Saccharomyces cerevisiae (yeast), rabbit reticulocyte, wheat germ and HeLA cells. Since there are many combinations of protein synthesis to choose from, an optimal solution when it comes to reaction time can be found. An alternative to cell extract CFPS is PURE (protein synthesis using recombinant elements). This system uses individual cleansed components instead of cell extract, but still contains all components needed, included translation factors which through manipulation is produced in a higher yield than normal and cleansed as needed.

This method is preferred in view of making it possible to control the gradient of the different components and one avoids also problems with proteases (proteins which breaks down peptide-bonds by hydrolysis).

### 2.4 A Description of the Process Involved in a CFPS/PURE System

The process in which DNA is translated into RNA via an RNA polymerase (ribonucleic acid) is called transcription. What differs RNA from DNA is i.a. a hydroxyl group at the 2′-position of the aldopentose, the ‘backbone’ of DNA/RNA, and the use of uracil instead of thymine bases. After the RNA polymerase translates DNA into mRNA (messenger RNA), ribosomes (which in this case, are located in the cytosol) pick up the sequence and, step by step, match it with rRNA (ribosomal RNA), and then recruit the matching amino acids with the help of tRNA (transfer RNA). ERNA consists of anticodons to mRNA that encode for a specific amino acid (hereafter referred to as AA). For example is mRNA code for lysine ‘AAG,’ and the tRNA linked to lysine is then ‘UUC’ and will be recruited if there is a match. Amino acids are linked to the existing chain until a stop codon is read.

“Exons and introns” are not necessary for CFPS/PURE and are therefore not explained.

RNA transcription can be divided into three parts; initiation, elongation and termination. Initiation occurs when RNA polymerase (RNAP) binds to a region of the DNA called a promoter. After finding a start codon, RNAP will separate the two strands of the DNA helix and transcription will begin. This happens by adding nucleotide 5′-triphosphates (NTP) to the 3′ end of the RNA chain. Transcription will therefore copy the 3′-5′ match of DNA. Elongation occurs in that NTP enters a separate NTP channel to RNAP, and the 3′-OH group attacks the alpha phosphate and uses the binding energy to add the base. Through this process, RNAP will form a short RNA/DNA hybrid to find a matching base and disconnect the next 17 base pairs (bp) in the DNA sequence. Termination occurs when RNAP reads a stop codon. The complex is not explained here with respect to the alpha, beta, sigma, and omega parts, but is described for simplicity as a holo-complex.

Ribosomal activity, translation, can be divided into three parts; initiation, elongation and termination. In initiation, the carboxyl group of each AA is activated in the cytosol by attaching it to a tRNA, a process that uses ATP and is assisted by the protein “aminoacyl tRNA synthase.” Synthesis initiation occurs in that mRNA binds to two ribosomal subunits and the first tRNA to be used in the synthesis. The first aminoacyl-tRNA complex binds to the mRNA codon AUG, which signals the beginning of the polypeptide. Elongation occurs when +ERNA-AA is brought to the complete ribosomal complex, its tRNA anticodon links to the mRNA sequence, it is placed next to the present tRNA-AA, and it links AA together before the original tRNA leaves the complex and the procedure can be repeated as needed. The binding of aminoacyl-tRNA to the ribosome and the movement of the ribosome along the mRNA sequence are driven by the hydrolysis of GTP and require elongation factors attached to the ribosome. Termination occurs when the ribosome encounters a stop codon. The polypeptide is then released from the complex, aided by proteins called “release factors,” and the ribosome can be reused in the next round.

For the protein to function as designed, post-transcriptional modifications are usually needed to “fold” into correct 3D modification. This often includes removing one or more amino acids, methylation, carboxylation, acetylation, or cleaving/adding oligosaccharides. In this patent, mainly self-folding proteins will be used, meaning they do not require additional modifications.

### 2.5 Different Types of Reporter Molecules

### 2.5.1 Introduction

The choice of reporter molecule will vary according to needs.

If the test is to be used outside of laboratories and without the presence of healthcare professionals, a reporter molecule that causes a color change will be used as it is easier to detect with the naked eye.

In a Point of Care test, β-galactosidase can be used, as a strong blue color will appear. The additional amplification step will contribute to the signal strength.

If the method is to be used in laboratories that need to test many samples simultaneously, it may be appropriate to use a fluorescent reporter molecule as this can reduce the requirement for protein concentration when UV transmission techniques are used.

### 2.5.2 BFP

Fluorescent proteins have been used in research for a long time to confirm successful transfection of genetic material by observing the distinct self-Illuminating colors that arise. Examples of such fluorescent reporter molecules can be BFP (blue fluorescent protein) or the related GFP (green fluorescent protein).

BFP has a higher capacity to refold than GFP (green fluorescent protein). This will affect how long the reaction can be detected, making BFP a better candidate as a reporter molecule. It will require some more resources in the form of increased concentrations of reaction components (e.g. tRNA/amino acids/ATP) since BFP is 29 kDa, while GFP is 28 kDa. However, it will likely still be advantageous given that it has a more stable structure.

2.5.3 β-Galactosidase Combined with x-Gal:

These two are primarily used to detect transgene expression in experimental animals, but since β-galactosidase has enzymatic activity, it can help increase the sensitivity of the test if an additional amplification step is needed. β-galactosidase will cause a strong amplification by cleaving the substrate 5-bromo-4-chloro-3-indolyl-β-D-galactopyranoside, also known as X-gal. After cleavage, X-gal will produce a strong blue color. The enzyme depends on many different chaperones to fold properly. If one were to choose to use the PURE system, this would be an additional cost. However, CFPS uses complete machineries that may already contain these components (chaperones).

### 2.6 Examples of Application of the Invention to Detect Gene Defects

The method can also be used to test for genetic defects, aiming to identify the occurrence of genetic disorders such as Huntington's disease. When applying the method for this purpose, a biopsy is taken from the patient, and it is tested for correct/healthy gene sequence,

A negative result indicates the presence of a gene defect.

### 2.7 Examples of Application of the Invention to Diagnose Unknown Infectious Diseases Based on Deduction from a Phylogenetic Tree

### 2.7.1 Method for Diagnosing Unknown Infectious Diseases

If CAPS is used with two reporter molecules which gives different color/frequency, it will be possible to deduce an unknown disease by using databases with genetic information. Examples of such databases are Genbank, Uniprot and Ensembl. Genetic information for microorganisms has also been categorized as phylogenetic trees, examples involve Phylofacts and Tree of Life.

This procedure will then entail a separation of different diseases based on conserved DNA/RNA sequences unique for a branch of the phylogenetic tree of a particular microorganism. By selecting two sequences in a step-by-step manner which separate a branch of a phylogenetic tree, one can in the end identify the disease-sequence without using symptoms for diagnose. In praxis a first test containing X number of reporter molecule sequences identify which main category the disease originates from (virus, bacteria etc). The next step will deduce which subtype of microorganism that causes the disease. The process is repeated as needed until one has identified the exact microorganism-given that one has reporter molecule for exactly this microorganism,

Two possible reporter molecule sequences that may be used are GFP and BFP (green/blue fluorescent protein, point 2.5.1). The test could e.g. indicate a viral infection by luminescing blue and a bacterial infection by luminescing green. Further steps will use similar methods.

BFP and GFP are examples of fluorescent that can be applied with the branching methods. Two fluorescent reporter molecules are used in the same test to identify which branch to continue with in the next round.

With machine reading it would be possible to diagnose multiple diseases in the same test, given that reporter molecules that are connected to CAPS have a big enough difference in absorbance frequency to be separated by, for instance, UV-spectroscopy.

The testing method is coinciding with the method described above.

In practical terms, this process could be automated in a way in which continuous tests are run down the phylogenetic tree for the same initial test, based on the outcome of the individual steps.

1. Method for detection of a microorganism in an organic sample in a container by a CRISPR/Cas-system cutting a pre-defined part of a microorganism genome (1) if the microorganism tested for is present in the test, characterized in that a nucleotide sequence coding for a reporter molecule (6) with a partly complete promotor element/start codon (8), is ligated with the cut part of the microorganism's genome thereby forming a complete promotor element or start codon (7, 8) that produces mRNA (11) by RNA polymerase (10), resulting in in a chain of amino acids (13) that can be detected and thereby verify the presence of the microorganism in the sample.

2. Method for detection of a microorganism in a test according to claim 1, characterized in that all components necessary for synthesis of a reporter molecule, except from the organic test, is present in the container before the organic sample is added.

3. Method for detection of a microorganism in a test according to claim 1 1, characterized in that the reporter molecule causes a change of colour.

4. Method for detection of a microorganism in a test according to claim 1, characterized in that the reporter molecule causes a change of light Intensity in the test.

5. Method for detection of a microorganism in a test according to claim 1, characterized in that the reporter molecule is produced through cell free protein synthesis.

6. Method for detection of an unknown microorganism in an organic sample by using one or several CRISPR/Cas-systems, characterized in sequentially identifying the unknown microorganism by combining the unknown sequence from the microorganism with main categories within a phylogenetic tree, and thereafter test the unknown gene sequence against other sequences in the under category, which is repeated until the microorganism is unequivocally identified.

7. Method for detection of a disease caused by a known gene mutation in an organic sample in a container by a CRISP/Cas-system cutting a pre-defined part of the cells genome (1) if the microorganism tested for is present in the sample, characterized in a DNA/RNA sequence (6) with a partly complete start codon (8)—where the sequence coding for a reporter molecule is ligated with the cut part of the microorganism's genome to form a complete promotor element and/or start codon (7,8), that further produces mRNA (11) by RNA polymerase (10), that results in in a chain of amino acids (13) that can be detected and thereby confirm the presence of the microorganism in the sample.
